(19) United States
(12) Patent Application Publication
(10) Pub. No.: US 2024/0035959 A1
(43) Pub. Date: Feb. 1, 2024

(54) SPECTROMETER

(71) Applicant: FRAUNHOFER-GESELLSCHAFT ZUR FÖRDERUNG DER ANGEWANDTEN FORSCHUNG E.V., München (DE)

(72) Inventors: Hendrik SCHLICKE, Hamburg (DE); Jan-Steffen NIEHAUS, Wentorf (DE)

(73) Assignee: FRAUNHOFER-GESELLSCHAFT ZUR FÖRDERUNG DER ANGEWANDTEN FORSCHUNG E.V., München (DE)

(21) Appl. No.: 18/268,111

(22) PCT Filed: Nov. 9, 2021
(86) PCT No.: PCT/EP2021/081056

§ 371 Date: Jun. 16, 2023

(30) Foreign Application Priority Data
Dec. 18, 2020 (DE) 10 2020 216 283.2

(51) Int. Cl. G01N 21/27; C09K 11/88; C09K 11/66

(52) U.S. Cl. CPC G01N 21/27; C09K 11/883; C09K 11/661; B82Y 20/00

(57) ABSTRACT

The present invention relates to a spectrometer comprising a plurality of arrays of quantum dots, the arrays being arranged such that primary light emitted by a light source is incident thereon and excites the quantum dots such that the quantum dots emit secondary light such that the secondary light can be incident on a sample, the secondary light having a different spectral distribution to the primary light, at least one detector configured to receive the secondary light reflected or transmitted by the sample, and an evaluation device configured to determine spectral information of the secondary light received by the at least one detector. The invention also relates to a method for producing such a spectrometer.

### CROSS REFERENCE TO RELATED APPLICATIONS

The present application is a U.S. National Phase application under 35 U.S.C. § 371 of International Patent Application No. PCT/EP2021/081056 filed Nov. 9, 2021, which claims priority of German Patent Application No. 10 2020 216 283.2 filed Dec. 18, 2020. The entire contents of which are hereby incorporated by reference.

### FIELD OF THE INVENTION

The present invention relates to a spectrometer.

### BACKGROUND

Spectrometers are used to determine the properties of various different objects. For instance, when analysing the chemical composition of e.g. everyday objects, food, waste, materials, raw materials, recyclable materials, arable soils and pharmaceuticals, an absorption or reflection spectrum needs to be determined. Spectroscopy furthermore offers numerous possibilities in medical diagnostics. In particular the near-infrared (NIR) wavelength range is highly relevant since inter alia characteristic and material-specific overtone and combination vibrations of molecular components occur in this range. The analysis of the absorption and reflection spectra defined by these vibrations inter alia allows conclusions to be drawn regarding the origin, degree of ripeness and quality of food or regarding the composition of the samples to be analysed, such as raw materials. Here and in the following, the term “absorption spectrum” is understood to mean a transmission spectrum, i.e. a spectrum of the light transmitted through the sample.

In the present application, the term “spectrum” refers to a distribution of the amplitude of the reflected or transmitted light as a function of the wavelength or frequency of this light. A wavelength-dependent or frequency-dependent resolution of the amplitude is hereby possible. In the context of a spectrometer, the wavelengths emitted by the light source used are thus resolved in the wavelength or frequency range. The reflection or transmission intensity to which the incident wavelengths correspond can accordingly be determined, although possibly not continuously but only for individual points.

Such absorption or reflection spectra are obtained by measuring the attenuation of the transmitted or reflected electromagnetic radiation of one or more specific wavelengths by the sample material. Conventional spectrometers are based on an adjustable monochromatic light source and a detector. The adjustable monochromatic light source usually consists of a broadband light source, such as a halogen or deuterium lamp, and a monochromator. The latter usually consists of one or more diffraction gratings and slits, whereby selection of the wavelength can be achieved by rotating the grating(s). However, the use of mechanical components means that it is difficult to miniaturise such a spectrometer.

In order to achieve miniaturisation, the use of a wavelength-selective detector array is, for instance, proposed. The pixels of the detector array are hereby provided, for example, with different filters that are transparent to different wavelengths. The transmitted or reflected light intensity at different wavelengths can thus be determined. Such spectrometers are described, for example, in US 2014/0061486 A1 or in U.S. Pat. No. 10,066,990 B2.

A further approach is to use a matrix of switchable light sources of different emission wavelengths. These may be, for example, different LEDs, the emission wavelengths of which result from the use of different semiconductor materials. The use of colour converters/phosphors to adjust the LED wavelength is also possible. Such methods are described, for example, in U.S. Pat. No. 10,458,845 B2, JP 2008/020380 A, U.S. Pat. No. 8,279,441 B2, US 2012/0327410 A1, and U.S. Pat. No. 7,839,301 B2. A similar method is also described in U.S. Pat. No. 10,041,833 B1.

Although the determination of spectra was mentioned above, a further field of use of spectrometers is the determination of spectral information. In the present application, “spectral information” is understood to mean that the wavelengths or frequencies incident on the sample may not necessarily be resolved in the output. For example, when determining the water content of a sample, it may be sufficient to measure the cumulative light absorption at a wavelength of 1950 nm and 1450 nm in order to draw conclusions about the water content of the sample. The term “spectral information” is inasmuch to be understood to mean properties that are based on measurements of the transmission or reflection properties of the sample, but do not necessarily enable wavelength or frequency resolution. The determination of a spectrum is thus a specific case of determining spectral information.

### SUMMARY

The present invention relates to a spectrometer that can be used to obtain absorption and reflection spectra or such spectral information by measuring light reflected or transmitted by a sample material. The spectrometer is configurable and easy to manufacture.

The invention is defined by claim 1. Preferred embodiments are defined in the dependent claims.

According to claim 1, a spectrometer comprises a plurality of arrays of quantum dots. Quantum dots (QDs) are nanoscopic material structures typically consisting of one or more semiconductor materials. Charge carriers (i.e. electrons and/or holes) have limited mobility in at least one spatial direction in QDs due to the small particle size. This limitation leads to a change in the optoelectronic properties, in particular the absorption and emission behaviour (“quantum confinement”). Quantum dots typically contain in the order of 103 to 105 atoms. Quantum dots may have various shapes, such as spheres with a diameter of 1 to 200 nm, preferably 2 to 100 nm, more preferred 2 to 50 nm. Shapes such as rods, tetrapods, nanowires or platelets are also possible. In these cases, at least one dimension must be subject to quantum confinement. In the case of core/shell structures, at least one dimension of the core must be subject to quantum confinement.

These multiple arrays of quantum dots function as colour converters and are arranged in the beam path of primary light such that primary light emitted by a light source can be incident thereon and excites these quantum dots with the result that the quantum dots emit secondary light. In other words, the quantum dots convert light emitted by a light source, whereby the spectral distribution of the light changes in that excitonic states in the quantum dots are excited and subsequently relax to emit secondary light. The secondary light has a different spectral distribution to the primary light. This secondary light can be incident on a sample and can then be used to determine the transmission or reflection behaviour of said sample such that the absorption or reflection spectrum thereof or, more generally, transmission or reflection spectral information can be obtained.

The spectrometer furthermore comprises at least one detector. Such a detector may, for example, be a photodiode or a photoconductive sensor (e.g. InGaAs, InAs, InSb, PbSe, PbS) or, for example, a CCD array capable of receiving secondary light reflected or transmitted by the sample. However, any other device that can receive the emitted light and that can be used to detect the intensity of the reflected or transmitted secondary light in a specific wavelength range is possible.

An evaluation device is furthermore provided, which is configured to determine spectral information of the secondary light received by the at least one detector. When recording a spectrum, this does not have to be continuous—it is sufficient if intensity values are determined for one or more discrete wavelengths.

The use of quantum dots to convert the light of a primary light source is advantageous in that it has been found that the emission wavelength of the quantum dots, and thus the conversion behaviour thereof from primary light to secondary light, can be almost infinitely adjusted by the choice of synthesis parameters, the composition thereof and in particular the particle size thereof. Such quantum dots that differ from one another in terms of spectra mean that it is possible to excite different wavelength ranges of the sample and thus to determine spectral information/a spectrum over a large range. As a rule, quantum dots can also be excited by photons of a broad wavelength range above their bandgap energy, but exhibit narrow-band emission, the wavelength of which corresponds approximately to the bandgap energy. This leads to a high degree of flexibility in the selection of suitable primary light sources for exciting the quantum dots.

Formulations of different quantum dots, optionally with suitable matrix materials, furthermore behave very similarly with respect to their deposition properties, as a result of which a high degree of configurability and flexibility is achieved when manufacturing the spectrometer. Quantum dot colour converter arrays with pixels of different emission wavelengths can thus be obtained in a cost-effective manner by applying different quantum dot segments. QDs of different sizes can furthermore be mixed so that individual pixels can also be obtained, the emission spectra of which contain, for example, multiple bands and are matched to the absorption spectra of any target analytes/mixtures.

Such a spectrometer can therefore be easily and flexibly configured during manufacture, thus allowing for greater flexibility. QDs furthermore usually have a high photoluminescence quantum yield and are superior to organic fluorophores, in particular in the NIR range. High conversion efficiencies and light intensities can thus be obtained by using QDs.

The spectrometer advantageously also comprises a light source arranged such that the primary light emitted thereby is incident on the arrays of quantum dots. While it is, in principle, possible for the spectrometer to use ambient light such as sunlight as its primary light, it is advantageous for the spectrometer to comprise a light source. This allows the spectrometer to be used regardless of whether ambient light is present and, if so, what type of ambient light is present. Such a light source also has a defined emission intensity and can be configured such that it is particularly suitable for exciting the quantum dots. If ambient light is used, a device is required to selectively admit ambient light to the arrays of QDs or, if all or a plurality of arrays of QDs are illuminated simultaneously, to selectively admit the emission thereof to the sample. Examples of such devices are LCDs, shutters or micro-opto-electro-mechanical components. These can be disposed between the array of QDs and the sample, or between the light source and the arrays of QDs, and thus selectively prevent light from the respective array from being incident on the sample.

It is furthermore preferred that at least two of the plurality of arrays of quantum dots have different emission spectra. This has the advantage that not only the transmission or reflection spectrum at a single wavelength is determined, but that this spectrum is also determined for a plurality of wavelengths (spectra recording). Such a transmission or reflection spectrum is significantly more informative. Different emission spectra is understood to mean that the peaks of the maximum intensity of the spectra are shifted relative to one another by at least 1 nm, preferably at least 10 nm, more preferred 50 nm, even more preferred 100 nm and most preferred 200 nm. Alternatively or additionally, the standard deviation of the peaks of the maximum intensity differs by at least 50 nm, preferably 100 nm.

It is furthermore preferred that the spectrometer is configured such that only one of the arrays of quantum dots with different emission spectra at a time emits secondary light such that it can be incident on the sample. In other words, this means that only one of the arrays leads to secondary light being incident on the sample. This allows the sample to be selectively illuminated with secondary light from one of the arrays, resulting in the ability to study the spectral behaviour of the sample relative to different excitation wavelengths/ranges. It is also possible for the light emitted by the individual arrays to be modulated such that they are no longer controlled in a “binary”, i.e. on-off, manner, but in an analogue manner. It can in particular be ensured that the light is modulated in a wave-like (e.g. sinusoidal) manner.

It is furthermore preferred that the light source comprises a plurality of sub-light sources, each of which is respectively coupled to one of the arrays of quantum dots such that light emitted by the respective sub-light source is incident (only) on the respective array. Thus, by switching the sub-light sources on or off or by modulating the sub-light sources, one of the arrays of quantum dots can be excited in a targeted manner. For this purpose, the control device is configured such that it can control from which of the sub-light sources primary light is emitted, as a result of which the described controllability can be achieved. Power consumption can thus also be reduced since light sources do not have to be operated unnecessarily. If, for example, LEDs or laser diodes, which can be easily operated in a pulsed mode or in an otherwise modulated mode, are used as primary light sources, a pulsed or modulated emission of the QD arrays can thus be realised very easily.

Alternatively or additionally, the light source (11, 111) may comprise a plurality of selectively light-transmissive windows. These are controlled by the control device such that they selectively allow light from a (single) primary light source to pass through. In this case, the primary light source is a light source that provides light to some (preferably: all) of the windows, which is then incident on the arrays. The light is allowed to pass through the windows and then impinges only on the corresponding array, exciting the QDs thereof. It is also possible to provide a plurality of selectively light-transmissive windows that are controlled by the control device such that they selectively allow secondary light from the arrays to pass through, the secondary light being generated by primary light from a single primary light source. The windows can be implemented by means of LCDs or shutters or micro-opto-electro-mechanical members. Only a single light source is thus required, as a result of which the complexity of the arrangement can be simplified.

As an alternative embodiment, it is preferred that the light source be completely external and independent of the spectrometer. For example, the light source may be ambient light or sunlight. It is, of course, also possible to use a specialised lamp as the light source. The primary light emitted by the external light source can then be incident on the arrays.

The spectrometer then furthermore comprises a device that controls from which of the arrays secondary light is incident on the sample. Such a device may, for example, comprise components configured to selectively prevent primary light from the light source from being incident on one or more of the arrays. As a result, it is possible to control on which of the arrays primary light is incident and which of the arrays is thus excited. Spectral information can thus be obtained by this selective control of the arrays. Alternatively or additionally, the device may comprise components configured to selectively prevent secondary light emitted by the arrays from being incident on the sample. Thus, in contrast to the embodiment described above, it is not the primary light but rather the secondary light that is blocked. This also allows spectral information to be obtained. The components for blocking the primary light and/or the secondary light preferably comprise shutters and/or LCD members and/or micro-opto-electro-mechanical members. Such components are easy to implement.

With a spectrometer that uses an external light source and thus does not require a built-in light source, the cost and energy consumption of the spectrometer can be reduced.

It is preferred that the particle sizes and/or particle compositions of the quantum dots in at least two of the plurality of arrays of quantum dots differ from one another. By using different particle sizes and/or particle compositions, the emission behaviour can be well controlled.

It is furthermore preferred that quantum dots having differing emission spectra are present in at least one of the plurality of arrays. This allows the emission of these arrays of quantum dots to be adapted, for example, to the absorption behaviour of analytes.

It is preferred that at least some of the quantum dots are configured such that they emit secondary light in the near-infrared range. The emission of such secondary light in the near-infrared range is of high practical relevance, as explained above.

The wavelength of the primary light is preferably shorter than that of the secondary light. Such a down-conversion is of particular practical relevance since in particular secondary light in the near-infrared range can be effectively produced as a result thereof.

The quantum dots are preferably embedded in a matrix. Such a matrix may consist, for example, of the ligands of the nanoparticles and/or of compounds suitable for cross-linking quantum dots. The quantum dots can alternatively/additionally preferably be incorporated in an organic matrix (e.g. a polymer matrix) or a matrix made of an inorganic material. This is particularly advantageous for the application of quantum dots.

Ligands of the quantum dots may, for example, be molecules carrying one or more functional groups with which they can bind to the surface of the quantum dots. Examples of such functional groups are thiols, disulphides, amimes, phosphines, phosphonic acids, carbamates, thiocarbamates, dithiocarbamates, carboxylic acids, polyethers, phosphine oxides, dihydroxyphenyl groups and nitrile groups.

Suitable compounds for cross-linking quantum dots include molecules that have a plurality of these functionalities and are sterically suitable for binding to more than one quantum dot. By selecting a suitable cross-linking compound, composites of quantum dots can be produced, the properties of which, such as the distance between the quantum dots, can be adjusted. Inorganic compounds such as metal chalcogenides can also be used as ligands/crosslinkers [Kovalenko et al. Science 2009, 324, 1417].

A wide variety of organic substances, such as polymers, which have high transparency to the primary light and the secondary light, can be used as the organic matrix material for incorporating the quantum dots. Examples include photoresists (e.g. SU-8), (UV-curable) adhesives such as Norland Optical Adhesive (NOA60, NOA61, NOA63, NOA65, NOA68, etc.), dendrimers, mercapto esters, thioesters, dithioesters, polythioesters, polydithioesters, polythiols, polythioethers, polymethyl methacrylate (PMMA), polystyrene (PS), polycarbonates, polyethylene terephthalate, polyurethanes, polypropylene, polyethylene, polyamides, polyethylene glycol, polylactides, polyimides, polyisoprene, polyethers, polyesters, as well as copolymers or mixtures thereof.

Inorganic compounds that are transparent to the primary and secondary light can be used as inorganic matrix materials. Examples include silicones, aluminium oxide, titanium oxides, silicates, indium tin oxide, silicon dioxide, so-called spin-on glass, zirconium dioxide, sodium fluoride, sodium yttrium fluoride, lanthanum phosphates, lanthanum phosphorus vanadate, lanthanum vanadate, yttrium vanadium phosphate, hafnium oxide, HSQ coatings, polydimethylsiloxane (PDMS), yttrium oxide, zinc oxide, silicon nitride as well as mixed phases.

Photostructurable (light-curable or light-destructible) matrix materials such as photocrosslinkable ligands or photoactive crosslinker molecules, suitable photoresists, or UV-curable adhesives have proven to be advantageous. These materials allow an easy (direct) photolithographic structuring of the arrays of quantum dots. Methods such as masking by means of photoresists followed by lift-off processes or etching processes can be used for structuring other materials.

To apply the arrays of quantum dots, formulations of quantum dots and matrix materials can be prepared, for example by introducing solvents. After deposition of the formulation, the solvent can then be removed, if necessary, to obtain the arrays of quantum dots.

The quantum dots can furthermore be introduced into formulations with precursor compounds of the matrix materials. After deposition, the precursor compounds of the matrix materials can produce the matrix material and thus the arrays of quantum dots by means, for example, of photo-induced or heat-induced reactions.

By selecting a suitable matrix material, optimal deposition properties (e.g. viscosities and processing temperatures of formulations/inks, compatibilities with surfaces, adhesion to surfaces) can be set for the use. In addition, the mechanical characteristics of the coating/composite can be adjusted by the selection of the matrix material. The matrix can in particular also protect (e.g. against (photo-)oxidation) the quantum dots against external influences (e.g. air, water).

The arrays of quantum dots are preferably arranged on a surface of the light source. Such a configuration is particularly advantageous since the positioning of the quantum dots relative to the light source is now fixed and can no longer change. The quantum dots, e.g. embedded in a matrix material, can thus be applied, for instance, directly to the LED chip or the LED housing.

It is furthermore possible to arrange the arrays of quantum dots on a transparent or non-transparent substrate, e.g. a glass substrate or a wafer, by means of lithography processes. This substrate can then, for example, be arranged as a multispectral colour conversion filter on an array of primary light sources or in the beam path of an array of primary light sources.

In addition, a suitable matrix material (e.g. a polymer) can itself be used as a support for the arrays of quantum dots. Quantum dot-containing films can, for example, be manufactured without a further substrate, which are placed as quantum dot-based colour converters on an array of primary light sources or in the beam path of an array of primary light sources.

It is furthermore preferred that the quantum dots in one or more of the arrays consist of a plurality of material domains, the material domains preferably having a core-shell configuration or a Janus configuration. Such quantum dots have proven to be particularly advantageous for use in spectrometers as emitter materials. Examples of core-shell configurations in quantum dots are described, for example, in [Jang et al., Chem. Commun. 2017, 53, 1002 to 1024] or WO 2014/033213 A2, the latter in particular disclosing manufacturing methods for such particles. Such core/shell quantum dots can consist, for example, of CdSe/CdS, ZnSe/ZnS, ZnTe/ZnSe/ZnS, CdSe/CdS/ZnS or InP/ZnS, InP/ZnSe, InP/ZnSe/ZnS and other combinations for the visible wavelength range or of PbSe/CdSe, PbSe/CdSe/CdSe [J. Am. Chem. Soc. 2017, 139, 32, 11081 to 11088], PbSe/CdSe/CdS, InAs/ZnS, InAs/ZnSe, InAs/ZnSe/ZnS, InSb/ZnS, InSb/ZnSe, InSb/ZnSe/ZnS, PbSe/PbS, PbTe/PbSe, PbTe/PbSe/PbS, PbS/PbO, PbSe/PbS/PbO, PbTe/PbSe/PbS/PbO and other combinations for the NIR wavelength range. It may additionally be advantageous if the core/shell materials do not have discrete transitions, but rather mixed phases between material domains. The particles may furthermore carry passivating shells made, for instance, of insulating materials such as oxides, e.g. silicon dioxide.

The primary light is incident on the plurality of arrays of quantum dots preferably in a modulated mode, even more preferred in a pulsed mode. Such modulated or pulsed incidence of the primary light can increase the sensitivity of the spectrometer since the use of a lock-in amplifier is possible. The primary light that is incident on different arrays of quantum dots may be modulated with different frequencies so that the secondary light emitted by these arrays of quantum dots is also modulated with these different frequencies. This has the advantage that light from different secondary light sources that is reflected or transmitted by the sample has modulations with different frequencies, and that the analysis of the spectral behaviour of the sample can thus take place by differentiating between the intensity signals measured in the frequency domain at the detector. A sinusoidal modulation has proven to be particularly easy to implement and analyse.

The quantum dots provided in at least one (preferably: all) of the plurality of arrays of quantum dots are preferably identical to one another. This leads to a great homogeneity of the secondary light which they emit. The term “identical” is understood here to mean that they are identical in terms of size and composition to the extent allowed by quantum dot manufacturing processes within manufacturing tolerances.

Individual arrays of quantum dots may furthermore also include mixtures of quantum dots of different sizes and/or compositions. This has the advantage that the spectral distributions of the light emitted by these arrays of quantum dots may contain a plurality of emission maxima that can be adapted to the absorption/reflection spectrum of one or more target analytes. As an extreme case, arrays of quantum dots emitting a broadband NIR spectrum can thus also be produced. Such light sources are difficult to realise in a conventional manner, e.g. with direct-emitting semiconductors.

The quantum dots in at least one of the arrays are preferably made from a semiconductor. This is preferably a IV-VI, II-VI, III-V or I-III-VI semiconductor and even more preferred a IV-VI semiconductor. Such semiconductors have led to particularly good conversion behaviour. Even more preferred, it is a semiconductor made of lead chalcogenides. Other materials that may be used for secondary light in the near-infrared range are PbS, PbSe, PbTe, InAs, InSb, InAsSb, InAsP, CuSe₂, CdTe, HgTe, SnTe, SnSe, InP, CuₓIn_{y}Te_{z}, CuₓIn_{y}Se_{z}, CuₓIn_{y}S_{z}, CuInSe₂, CuInS₂, AgₓIn_{y}Te_{z}, AgₓIn_{y}Se_{z}, AgₓIn_{y}S_{z}, AgInSe₂, AgInS₂, and for secondary light in the visible range are SnSe, SnS, CdSe, CdS, ZnS, InP, CuₓIn_{y}Se_{z}, CuₓIn_{y}S_{z}, CuInSe₂, CuInS₂, AgₓIn_{y}Se_{z}, AgₓIn_{y}S_{z}.

It is furthermore preferred that a method for producing a spectrometer according to any one of the preceding claims produces the arrays of quantum dots by way of spin coating, dip coating, drop coating or a printing process. It is particularly preferred, in the case of an arrangement of arrays of quantum dots to be produced, that spin coating and dip coating be performed in conjunction with lithographic structuring. Such methods are well characterised and established, and thus lead to a homogeneous, controlled deposition and to a spectrometer with good properties as regards spectral analysis.

### BRIEF DESCRIPTION OF THE DRAWINGS

FIG. 1 schematically shows a spectrometer according to a first embodiment of the invention.

FIG. 2 shows the emission behaviour of quantum dots.

FIG. 3 shows a more detailed configuration of the spectrometer of FIG. 1.

FIG. 4 schematically shows a spectrometer according to a second embodiment of the present invention.

### DETAILED DESCRIPTION OF THE DRAWINGS

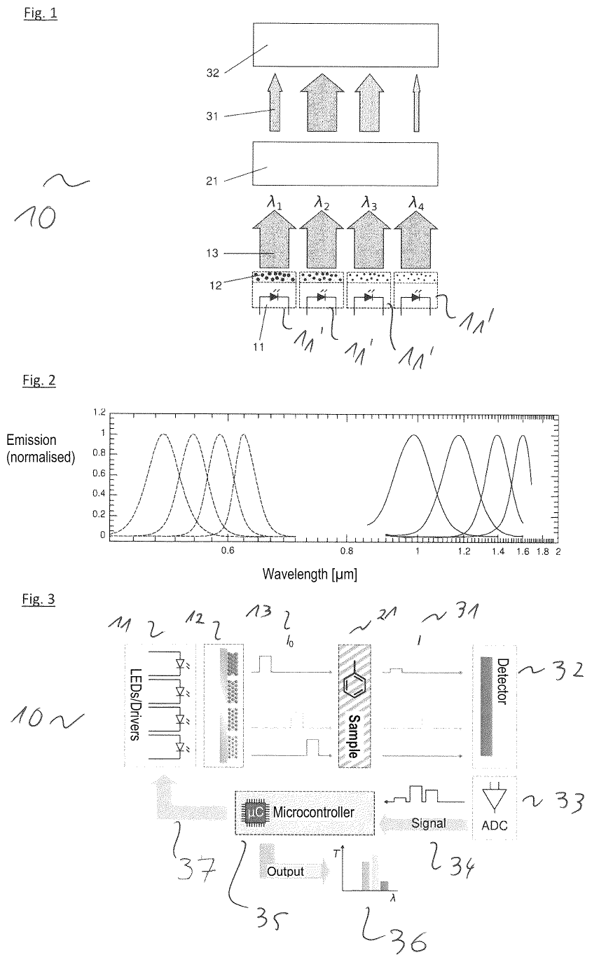

FIG. 1 and FIG. 3 schematically show an absorption spectrometer 10 according to a first embodiment of the present invention. A light source 11, which in the present embodiment takes the form of four sub-light sources 11′ in the form of LEDs, is provided in this absorption spectrometer. It should be noted that the number of sub-light sources is not limited. Deflectable lasers or VCSELs could, for example, also be used instead of LEDs. This light source 11 emits primary light in a wavelength range from UV to near-infrared light, i.e. a wavelength in the range from 200 to 3500 nm, preferably 300 to 2000 nm, more preferred 350 to 1100 nm.

The primary light emitted by these sub-light sources 11′ is incident on arrays 12 of quantum dots arranged at the sub-light sources 11′. As is schematically shown in FIG. 1, the quantum dots in the arrays are substantially identical, but differ in diameter and thus have different colour conversion behaviour. The quantum dots in the arrays 12 are thus excited by the primary light from the light source 11 and then relax to emit secondary light 13. The secondary light 13 typically has a longer wavelength than the primary light, which is referred to as down-conversion. However, it is, in principle, also possible for an up-conversion to take place and for the secondary light 13 to thus have a shorter wavelength than the primary light. The wavelength of the secondary light 13 is typically in the range of 300 to 5000 nm, preferably 390 to 3500 nm, more preferred 450 to 2500 nm. Owing to the different particle sizes of the quantum dots in the arrays 12, the wavelengths λ₁ to λ₄ of the secondary light 13 emitted by the arrays 12 differ.

The dependence of the wavelength of the emitted secondary light 13 on the size of the quantum dots is also shown in FIG. 2. This figure shows emission spectra of CdSe-based quantum dots (dashed lines) and of PbS-based quantum dots (solid lines), each for different particle sizes. The Y-axis is normalised so that the maximum value of the emission is 1 in each case. As is apparent from FIG. 2, the emission behaviour of the quantum dots is strongly influenced by changing the particle size.

The secondary light 13 is then incident on the sample 21 to be analysed. The light 31 transmitted by the sample is then detected by the detector 32. By switching the sub-light sources 11′ on or off in a targeted manner, it is thus possible to determine the absorption behaviour of the sample 21 with respect to secondary light 13 having different wavelengths λ₁ to λ₄, as a result of which spectral information or, in particular, the transmission spectrum of the sample 21 can be determined. This makes it possible to determine the properties of sample 21. The detector 32 may be a broadband detector using a semiconductor sensor, whereby such a semiconductor sensor may be based, for example, on CdS, CdSe, PbS, PbSe, InAs, InGaAs, InSb, HgTe. An intensity spectrum is recorded in the time domain by sequentially switching on and off or modulating one or more of the sub-light sources 11′.

A more detailed depiction of the spectrometer of FIG. 1 can be seen in FIG. 3, wherein a pulsed and staggered control of the sub-light sources 11′ is used. Since the sub-light sources 11′ are operated in pulsed mode, the secondary light 13 emitted by the arrays 12 is also emitted in a pulsed manner, which then leads to the transmitted light 31 also being emitted in a pulsed manner and thus being detected by the detector 32 in a pulsed manner. This makes it possible to activate the arrays 12 sequentially. The output of the detector 32 is converted into a digital signal 34 via an amplifier and an analogue-to-digital converter (ADC) 33, which is in turn emitted, for example, to a microcontroller, a field-programmable gate array (FPGA) or a digital signal processor (DSP) 35. This now outputs spectral information, specifically the spectrum 36, which is the end result of this analysis. The microcontroller/DSP/FPGA 35 also serves to emit control signals 37 to the light source 1 and the sub-light sources 11′. The microcontroller/DSP/FPGA can furthermore interpret the spectrum and display any information regarding the sample properties to the user. The spectrometer can furthermore be coupled to a computer or network and be used, for example, to monitor (automated) processes.

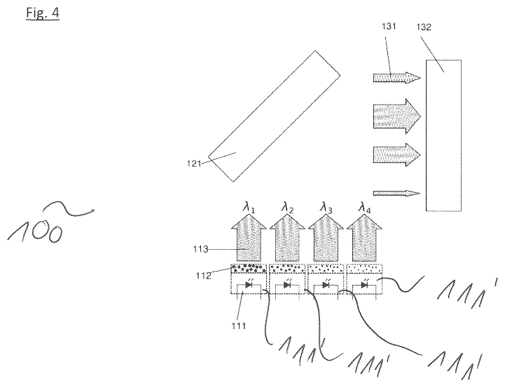

A further embodiment is shown in FIG. 4. As far as control and analysis are concerned, this embodiment functions in substantially the same manner as the spectrometer shown in FIGS. 1 and 3, which is why a description of the identical aspects is not provided here. Substantially the same reference numbers as in FIG. 1 are used here, with 100 being added in each case.

The spectrometer 100 shown in FIG. 4 is a reflectance spectrometer that records a reflection spectrum of the sample 121. It should be noted that neither the angle of incidence of the secondary light on the sample nor the exit angle are fixed and are arbitrarily selected. The same also applies to the other dimensions and angles in the embodiments. It should furthermore be noted that (for instance in the case of scattering on a rough surface) the angle of incidence and the angle of reflection do not have to be identical. A light source 111 consists of a plurality of (in the present case: four) sub-light sources 111′, on each of which an array 112 of quantum dots is provided. Light emitted by the sub-light sources 111′ is incident on these arrays 112, where it is converted into secondary light 113 that differs in wavelengths λ₁ to λ₄ from the wavelengths emitted by the four different sub-light sources 111′. The secondary light 113 is incident on the sample 121 and is reflected there. The reflected light 131 then impinges on the detector 132. The light or light intensity is measured there, so that a reflection spectrum can be created in the same way as described above in connection with FIG. 3. The spectrometer according to the second embodiment of the invention, as schematically shown in FIG. 4, can thus generate a reflection spectrum.

There are, in general, various advantages to using quantum dots. For example, as is also shown in FIG. 2, a size-quantisation effect occurs when using nanoscopic semiconductor particles, i.e. quantum dots. The band gap of the semiconductor material depends on the particle size. The electronic transitions leading to photoluminescence are thus also dependent on this particle size. Since nanoparticles can essentially now be produced in any size, it is therefore also possible to adjust the emission wavelength of the quantum dots almost infinitely. This is a significant advantage over the use of conventional inorganic or organic phosphors and emitters.

Quantum dots furthermore have a large Stokes shift. This means that particles with different emission wavelengths of the secondary light can be excited with primary light of the same wavelength. This simplifies the structure of the light source since a homogeneous LED array, for example, can be used as the primary light source.

The quantum dots can also be applied in the form of inks using comparatively simple processes such as spin coating, dip or drop coating, or printing processes based on inkjet printing on the primary light emitter or on a suitable substrate that can be applied to a primary light emitter.

The quantum dots with different emission wavelengths also behave substantially similarly with respect to the envisioned coating processes. The selection of the nanoparticles and the emission wavelengths and thus the analysis wavelengths of the spectrometer can thus be adapted for the given case of use without having to intervene heavily in the production process. The spectrometers can therefore be easily adapted to the issue to be examined.

Due to the similar behaviour of different QDs, the QD arrays may also consist of mixtures of QDs so that the emission behaviour thereof resembles the absorption behaviour of potential target analytes, and these can be detected more efficiently. It is extremely difficult to generate multi-band emission spectra with individual conventional LEDs or lasers. The use of QD arrays with multiple matched emission bands thus leads to savings in terms of space/cost and resources.

Quantum dots furthermore have a high photoluminescence quantum yield of up to 1, which results in high light intensity. Quantum dots are thus superior to organic fluorophores, in particular in the near-infrared range.

There are therefore numerous advantages which lead to a spectrometer such as shown in FIGS. 1, 3 and 4 being able to be used in a wide range of applications. These include in particular the identification, analysis and quality assurance of food, products, materials, raw materials, arable soils and waste products, etc. Such a spectrometer can furthermore also be used in medical diagnostics. Due to the possible miniaturisation, portable and configurable spectrometers can be developed which a user can use on site. It is also possible, in principle, to incorporate such a miniaturised spectrometer into a portable device such as a tablet or mobile phone. Even though the described spectrometers lead to advantages in particular in the near-infrared range, they can also be used in the visible and ultraviolet range.

1. A spectrometer comprising:
a plurality of arrays of quantum dots, the arrays being arranged such that primary light emitted by a light source is incident thereon and excites the quantum dots such that the quantum dots emit secondary light such that the secondary light can be incident on a sample, the secondary light having a different spectral distribution to the primary light,
at least one detector configured to receive the secondary light reflected or transmitted by the sample, and
an evaluation device configured to determine spectral information, preferably a spectrum, of the secondary light received by the at least one detector.

2. The spectrometer according to claim 1, further comprising a light source arranged such that primary light emitted by said light source is incident on the arrays of quantum dots.

3. The spectrometer according to claim 1, wherein at least two of the plurality of arrays of quantum dots have different emission spectra, the particle sizes and/or the materials of the quantum dots in at least two of the plurality of arrays of quantum dots preferably differing from one another.

4. The spectrometer according to claim 3, wherein the spectrometer is configured such that the secondary light emitted by the arrays of quantum dots with different emission spectra can be modulated, with preferably only one of the arrays of quantum dots with different emission spectra at a time emitting secondary light such that it can be incident on the sample.

5. The spectrometer according to claim 4, further comprising a control device for controlling from which of the arrays of quantum dots secondary light is incident on the sample.

6. The spectrometer according to claim 5, further comprising a light source arranged such that primary light emitted by said light source is incident on the arrays of quantum dots,
wherein the light source comprises a plurality of sub-light sources, each of which is respectively coupled to one of the arrays of quantum dots such that light emitted by the respective sub-light source is incident on the respective array, the control device being configured such that it can control from which of the sub-light sources primary light is emitted, or
wherein the light source comprises a plurality of selectively light-transmissive windows controlled by the control device such that they selectively allow light from a single primary light source to pass through, the light that is allowed to pass through the windows respectively being substantially incident on only one single corresponding array, and/or
wherein a plurality of selectively light-transmissive windows are provided that are controlled by the control device such that they selectively allow secondary light from the arrays to pass through, the secondary light being generated by primary light from a single primary light source,
wherein the windows preferably comprise LCDs and/or shutters and/or micro-opto-electro-mechanical members.

7. The spectrometer according to claim 1,
wherein the spectrometer is configured such that primary light from a light source external to the spectrometer can be incident on the arrays,
wherein the spectrometer furthermore comprises a device configured to control from which of the arrays secondary light is incident on the sample,
wherein the device preferably comprises components that are configured to selectively prevent primary light from the light source from being incident on one or more of the arrays and/or wherein the device preferably comprises components configured to selectively prevent secondary light emitted by the arrays from being incident on the sample,
wherein the components even more preferably comprise shutters and/or LCD members and/or micro-opto-electro-mechanical members.

8. The spectrometer according to claim 1, wherein quantum dots having differing emission spectra are present in at least one of the plurality of arrays.

9. The spectrometer according to claim 1, wherein at least some of the quantum dots are configured such that they emit light in the near-infrared range.

10. The spectrometer according to claim 1, wherein the wavelength of the primary light is shorter than the wavelength of the secondary light.

11. The spectrometer according to claim 1, wherein the quantum dots are embedded in a matrix, preferably a polymer matrix or a matrix made of an inorganic material.

12. The spectrometer according to claim 2, wherein the quantum dots are arranged on a surface of the light source.

13. The spectrometer according to claim 1, wherein the quantum dots of one or more of the arrays consist of a plurality of material domains, the material domains preferably having a core-shell configuration or a Janus configuration.

14. The spectrometer according to claim 1, wherein the spectrometer is configured such that primary light is incident on the plurality of arrays of quantum dots in a modulated, preferably pulsed, mode.

15. The spectrometer according to claim 1, wherein the quantum dots provided in at least one of the arrays of quantum dots are identical to one another.

16. The spectrometer according to claim 1, wherein the quantum dots in at least one of the arrays consist of quantum dots made of a semiconductor, preferably a IV-VI, II-VI, III-V or I-III-VI semiconductor, more preferred a IV-VI semiconductor, even more preferred a lead chalcogenide or lead sulphide.

17. A method for producing a spectrometer according to claim 1, wherein the arrays of quantum dots are produced by way of spin coating, dip coating, drop coating or a printing process.
